(19) United States
(12) Patent Application Publication
(10) Pub. No.: US 2026/0053607 A1
(43) Pub. Date: Feb. 26, 2026

(54) IMPROVED HANDLE ASSEMBLY FOR A DENTAL OR MEDICAL TOOL

(71) Applicant: Innovative Product Brands, Inc., Highland, CA (US)

(72) Inventor: Bryan Tapocik, Highland, CA (US)

(73) Assignee: Innovative Product Brands, Inc., Highland, CA (US)

(21) Appl. No.: 19/024,725

(22) Filed: Jan. 16, 2025

Related U.S. Application Data
(60) Provisional application No. 63/552,526, filed on Feb. 12, 2024.

(51) Int. Cl. A61C 17/02; A61C 17/022

(52) U.S. Cl. CPC A61C 17/0217; A61C 17/022

(57) ABSTRACT

A handle for attachment to a source selected from the group consisting of suction, air, and liquid at a rear end and attached to a tool selected from a dental tool and a medical tool at a front end, the handle has a handle valve body. A channel having a first open end and a second open end runs along a length of the body. The first open end is at an opposite end of the handle valve body from the second open end. A spherical chamber is formed within the channel and has openings at each transverse end. An indentation is formed in a top surface of the body and extends across a width of the handle valve body, wherein the spherical chamber extends up from the indentation. A ball valve is positioned within the spherical chamber. The ball valve is comprised of a ball, a ball channel running through a center of the ball and having a pair of open ends; a pair of spindle arms, wherein each spindle is in axial alignment and located on opposite sides of the ball; a socket formed in each spindle arm; a handle having a “U” shaped body coupled to the ball; and a pin member formed at the end of each leg of the “U” shaped body, each pin inserted into a corresponding socket securing the handle to the ball.

### CROSS REFERENCE TO RELATED APPLICATIONS

This application claims the benefit of U.S. Provisional Application No. 63/552,526, which was filed on Feb. 12, 2024 and titled “IMPROVED HANDLE ASSEMBLY FOR A DENTAL OR MEDICAL TOOL.” The entire contents of the foregoing are hereby incorporated in full by reference. This patent application is also related to U.S. Pat. No. 11,311,361, which issued on Apr. 26, 2022, was titled “HANDLE TO RETAIN A SOURCE OF SUCTION OR LIQUID AT ONE END AND A DENTAL TOOL OR MEDICAL TOOL AT AN OPPOSITE END AND METHOD OF SONIC WELDING OR ULTRASONIC WELDING OF A CONTAINER FOR THE HANDLE,” and was filed in the name of the same inventor herein. The entire contents of the foregoing are also hereby incorporated in full by reference.

### TECHNICAL FIELD

The present invention relates to the field of medical devices, and in particular, to a switching unit on a suction handle assembly to control a source of suction to the medical device to suck and remove a liquid from a patient's body during a medical procedure. The present invention also involves controlling a source of air or water to blow or spray debris from the patient's body during the medical procedure.

### BACKGROUND OF THE INVENTION

Earlier embodiments of a handle assembly used in conjunction with medical suction apparatuses exist. As may be shown in FIG. 1, an exploded view of a prior art embodiment of a handle assembly 100 may be seen. The handle assembly 100 may have a rear attachment 102, a handle valve body 104, a valve assembly 106, an O-ring 108, a front attachment 110 and a clip assembly 112. The handle valve body 104, O-ring 108 and valve assembly 106 are assembled before sonic welded to the front attachment 110 and to the rear attachment 102. The handle valve body 104 may protrude out the back side of the rear attachment 102 to allow for a hose to be attached. The hose can either be connected to a port that supplies suction, air, or water depending on the required use. The clip assembly 112 is securely attached to the front attachment 110 via a sonic weld or an ultrasonic weld. The clip assembly 112 may be used to secure different attachments to the handle assembly 100.

The handle assembly 100 is designed to be able to control the suction power or amount of suction with one's thumb via the valve assembly 106. By advancing the suction handle from the “off” position to the “on” position, the user can adjust the air flow. However, greater control of the suction power may be desired.

Therefore, it would be desirable to provide a system and method that overcomes the above.

### SUMMARY OF THE INVENTION

This summary is provided to introduce a selection of concepts in a simplified form that are further described below in the DETAILED DESCRIPTION OF THE INVENTION. This summary is not intended to identify key features of the claimed subject matter, nor is it intended to be used as an aid in determining the scope of the claimed subject matter.

The present invention is an improved handle for retaining a source of suction or a source of water at a rear end and retaining a dental tool at a front end. The handle includes several improvements in both the apparatus and construction of the apparatus.

In accordance with another embodiment of the present invention, a handle for attachment to a source selected from the group consisting of suction, air, and liquid at a rear end and attached to a tool selected from a dental tool and a medical tool at a front end is disclosed. The handle for attachment has an elongated handle valve body defining a channel. The channel runs through a length of the elongated handle valve body. A spherical chamber is defined by the elongated handle valve body and is positioned within a path of the channel. A ball valve is rotatably positioned within the spherical chamber.

In accordance with another embodiment of the present invention, a handle for attachment to a source selected from the group consisting of suction, air, and liquid at a rear end and attached to a tool selected from a dental tool and a medical tool at a front end is disclosed. The handle for attachment has an elongated handle valve body defining a channel. The channel runs through a length of the elongated handle valve body. A V-shaped indentation is formed in a top surface of the elongated handle valve body. A spherical chamber is defined by the elongated handle valve body and extends upwardly from the V-shaped indentation. A pair of side openings is formed on opposite sides of the spherical chamber. A ball valve is rotatably positioned within the spherical chamber. The ball valve has a ball defining a ball channel and the ball channel runs through a center of the ball. A pair of spindle arms is formed on the ball. The spindle arms are located on opposite sides of the ball and extend out of the pair of side openings. A handle is coupled to the spindle arms. The handle rotates the ball about the spindle arms.

In accordance with one embodiment of the present invention, a handle for attachment to a source selected from the group consisting of suction, air, and liquid at a rear end and attached to a tool selected from a dental tool and a medical tool at a front end is disclosed. The handle for attachment has an elongated handle valve body. A channel having a first open end and a second open end runs along a length of the elongated handle valve body, the first open end is at an opposite end of the elongated handle valve body from the second open end. A spherical chamber is defined by the elongated handle valve body and positioned within a path of the channel and has openings at each transverse end. An indentation is formed in a top surface of the elongated handle valve body and extends across a width of the elongated handle valve body. The spherical chamber extends upwardly from the indentation. A ball valve is positioned within the spherical chamber. The ball valve has a ball. A ball channel runs through a center of the ball and has a pair of open ends. A pair of spindle arms is formed on the ball, wherein each spindle arm is in axial alignment and located on opposite sides of the ball. A socket is formed in each spindle arm. A handle having a “U” shaped body is coupled to the ball. A pin member is formed at an end of each leg of the “U” shaped body. Each pin is inserted into a corresponding socket securing the handle to the ball.

Further novel features and other objects of the present invention will become apparent from the following detailed description and discussion.

### BRIEF DESCRIPTION OF THE DRAWINGS

The present application is further detailed with respect to the following drawings. These figures are not intended to limit the scope of the present application but rather illustrate certain attributes thereof. The same reference numbers will be used throughout the drawings to refer to the same or like parts.

FIG. 1 shows an exploded view of the components of a prior art handle assembly to retain a dental or medical tool at one end and a source of suction, air, or liquid at an opposite end;

FIG. 2 is an elevated perspective view of an exemplary embodiment of a handle valve body used in a handle assembly in accordance with an embodiment of the present disclosure;

FIG. 3 is a top view of an exemplary embodiment of the handle valve body of FIG. 2 in accordance with an embodiment of the disclosure;

FIG. 4 is a bottom view of an exemplary embodiment of the handle valve body of FIG. 2 in accordance with an embodiment of the disclosure;

FIG. 5 is a right-side view of an exemplary embodiment of the handle valve body of FIG. 2 in accordance with an embodiment of the disclosure;

FIG. 6 is a front view of an exemplary embodiment of the handle valve body of FIG. 2 in accordance with an embodiment of the disclosure;

FIG. 7 is another elevated perspective view of an exemplary embodiment of the handle valve body of FIG. 2 with dotted lines showing interior features of the handle valve body in accordance with an embodiment of the disclosure;

FIG. 8 is another top view of an exemplary embodiment of the handle valve body of FIG. 2 with dotted lines showing interior features of the handle valve body in accordance with an embodiment of the disclosure;

FIG. 9 is another right-side view of an exemplary embodiment of the handle valve body of FIG. 2 with dotted lines showing interior features of the handle valve body in accordance with an embodiment of the disclosure;

FIG. 10 is another front view of an exemplary embodiment of the handle valve body of FIG. 2 with dotted lines showing interior features of the handle valve body in accordance with an embodiment of the disclosure;

FIG. 11 is a cross-sectional side view of an exemplary embodiment of the handle valve body of FIG. 2 with the ball valve in an open position in accordance with an embodiment of the disclosure;

FIG. 12 is a cross-sectional side view of an exemplary embodiment of the handle valve body of FIG. 2 with the ball valve in a closed position in accordance with an embodiment of the disclosure;

FIG. 13 is an exploded view of an exemplary embodiment of the handle valve body of FIG. 2 in accordance with an embodiment of the disclosure; and

FIG. 14 is another exploded view of an exemplary embodiment of the handle valve body of FIG. 2 with dotted lines showing interior features in accordance with an embodiment of the disclosure.

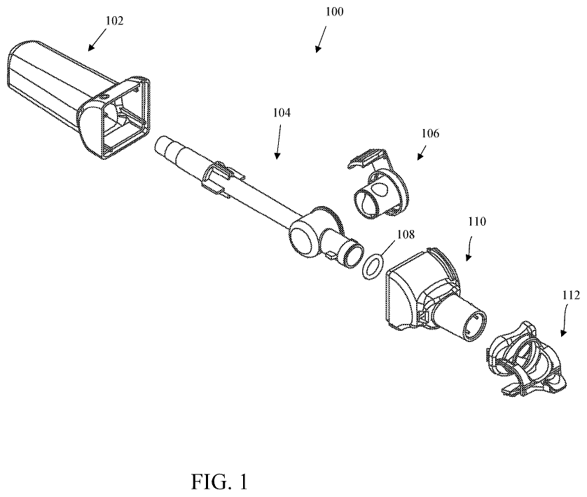

### DETAILED DESCRIPTION OF THE INVENTION

The description set forth below is intended as a description of presently preferred embodiments of the disclosure and is not intended to represent the only forms in which the present disclosure can be constructed and/or utilized. The description sets forth the functions and the sequence of steps for constructing and operating the disclosure. It is to be understood, however, that the same or equivalent functions and sequences can be accomplished by different embodiments that are also intended to be encompassed within the spirit and scope of this disclosure.

This patent application discloses an elongated handle valve body 200 designed to be able to control a suction, air supply, or liquid which is attached to the handle valve body 200. The handle valve body 200 has a ball valve 210. The ball valve 210 allows a user to rotate the ball valve 210 more easily from the “off” position to the “on” position and vice versa to adjust the suction level, air flow or liquid flow that is attached to the handle valve body 200.

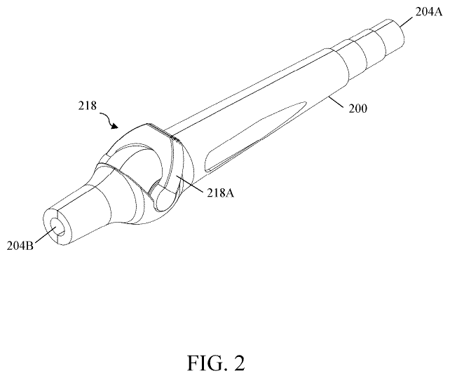

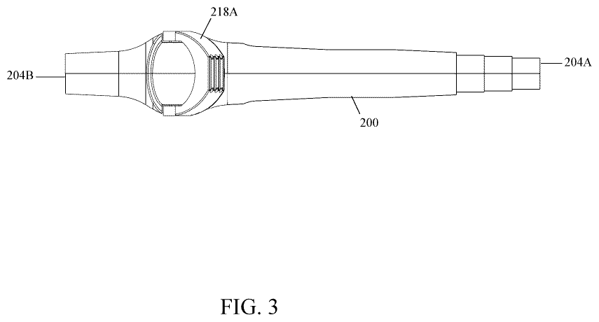

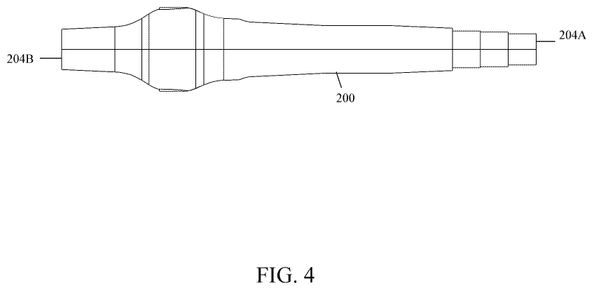

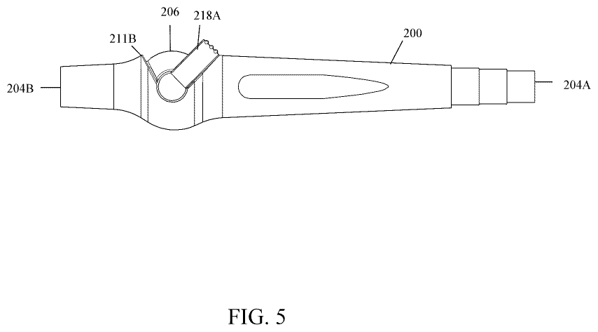

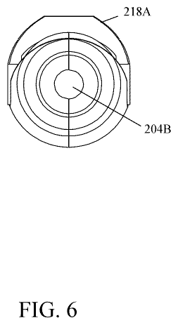

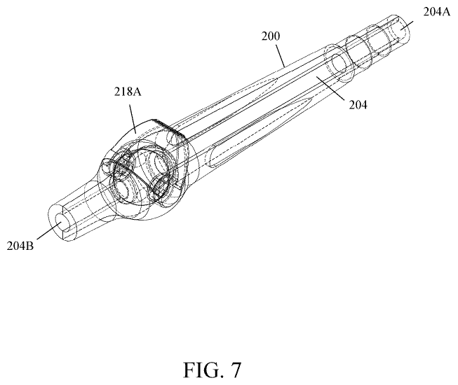

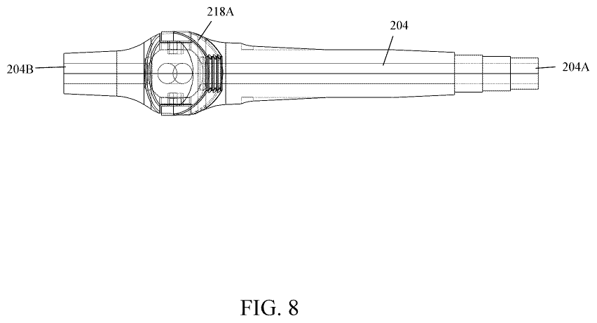

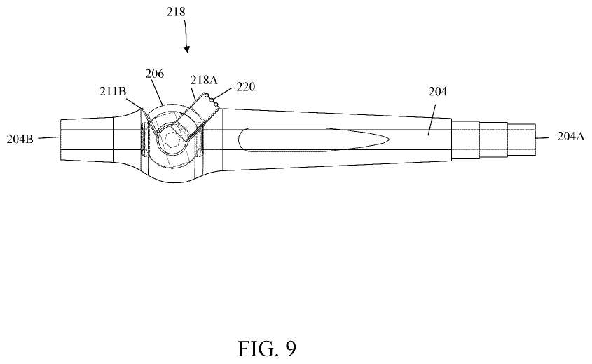

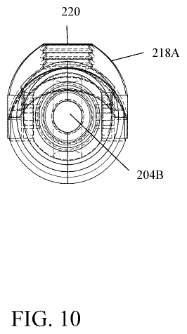

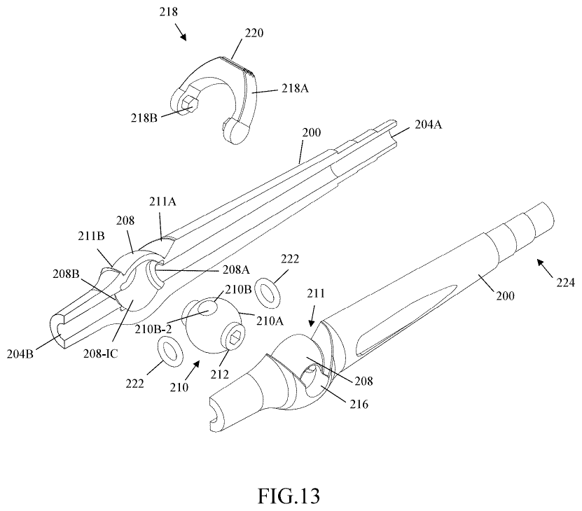

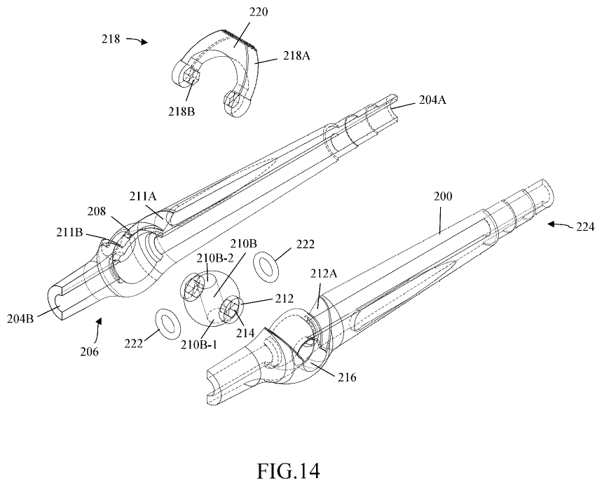

Referring to FIGS. 2-14, a handle valve body 200 (hereinafter body 200) may be shown. Located within an interior of the body 200 is a channel 204 having a first open end 204A and a second open end 204B. The channel 204 is defined by the interior of the body 200 and it may run along a length of the body 200 such that the first open end 204A is at an opposite end of the body 200 from the second open end 204B. In accordance with an embodiment, the channel 204 may run planar along a center line of the length of the body 200. The channel 204 may allow a source of suction, air, or a liquid to flow through the body 200 in either direction.

Within the path of the channel 204, the body 200 defines a spherical chamber 206. The spherical chamber 206 may be positioned proximate the second open end 204B of the channel 204. The spherical chamber 206 includes a transverse spherical wall 208 surrounding an interior chamber 208-IC with openings at each transverse end 208A and 208B. The openings at each transverse end 208A and 208B are contiguous with the interior chamber 208-IC. The opening at the transverse end 208A of the transverse spherical wall 208 is also contiguous with the channel 204 of the elongated body 200 and oriented toward the direction of the first open end 204A of the channel 204. The opening at the transverse end 208B of the transverse spherical wall 208 is also contiguous with the channel 204 of the elongated body 200 and oriented toward the direction of the second open end 204B of the channel 204.

An indentation 211 may be formed in a top surface of the body 200. The indentation 211 may be “V” shaped having two angled side walls 211A and 211B. The two angled side walls 211A and 211B of the indentation 211 together create a gap along the length of the top surface of the body 200 and the transverse spherical wall 208 of the spherical chamber 206 bridges that gap. A bottom area of the indentation 211 may be formed near a center line of the body 200. The indentation 211 may have a width that extends across the width of the body 200. The transverse spherical wall 208 extends upwardly from the indentation 211. In accordance with one embodiment, the transverse spherical wall 208 extends up from a lower area of the indentation 211.

The body 200 may have a ball valve 210. The ball valve 210 is formed of ball 210A having a ball channel 210B. The ball channel 210B runs through the ball 210A to form openings 210B-1 and 210B-2 through the ball 210A. In accordance with an embodiment, the ball channel 210B runs through a center line of the ball 210A.

The ball 210A has a pair of spindle arms 212 formed thereon. The spindle arms 212 are in axial alignment with one another wherein one spindle arm 212 is located on an opposite side of the ball 210A from the other spindle arm 212. In accordance with an embodiment, each of the pair of spindle arms 212 are formed on the surface of the ball and are positioned +/−90° from the openings 210B-1 and 210B-2. A socket 214 may be formed in each spindle arm 212. The socket 214 may be a multi-sided socket 214. For example, the socket 214 may be a hexagonal shaped socket, an octagonal shaped socket, or similar shaped socket.

The body 200 may define a pair of side openings 216 formed within the body 200. In accordance with an embodiment, the openings 216 may be formed in a bottom area of the indentation 211 and may be in axial alignment with one another. The side openings 216 may be positioned on opposite sides of the spherical chamber 206. The side openings 216 may extend outwardly and laterally in opposite directions from the transverse spherical wall 208 of the spherical chamber 206.

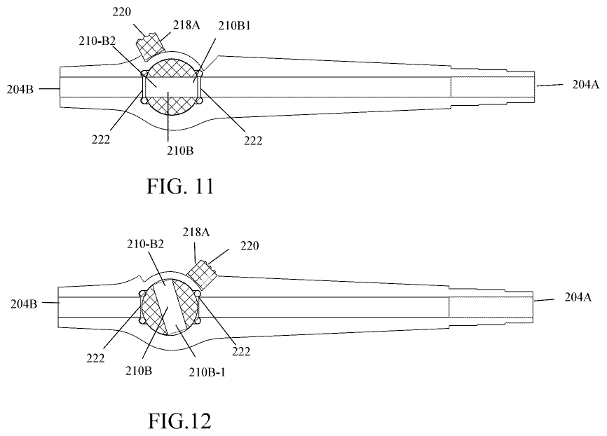

The ball valve 210 may be positioned within the spherical chamber 206 and may be rotatable within the spherical chamber 206. When positioned within the spherical chamber 206, each spindle arm 212 may extend out of one of the pair of openings 216 formed within the body 200. In accordance with an embodiment, each of the pair of openings 216 are positioned opposite from each other and are positioned at +/−90° from the openings at each transverse end 208A and 208B of the transverse spherical wall 208. In accordance with at least one embodiment, 90° is preferable. This may allow the ball valve 210 to rotate about the spindle arms 212 within the spherical chamber 206. When the ball valve 210 is properly positioned within the spherical chamber 206, the ball valve 210 may be rotate in one direction, for example counterclockwise. As shown in FIG. 11, when the ball valve 210 is in the “open” position, the ball channel 210B of the ball valve 210 aligns with and is contiguous with the channel 204 of the body 200, thus allowing a flow of air or liquid through the channel 204 and through the ball channel 210B. According to at least one embodiment, the diameter of the channel 204 may be equal to the diameter of the ball channel 210B. As shown in FIG. 12, when the ball valve 210 is rotated in an opposing direction, for example clockwise, the ball valve 210 is in the “closed” position. When the ball valve 210 is in the “closed” position, the ball channel 210B of the ball valve 210 is no longer aligned with the channel 204 of the body 200; instead, the ball valve 210 now blocks the flow of air or liquid through the channel 204.

A handle 218 may be attached to the ball valve 210. The handle 218 may be used to move the ball valve 210 to control the amount of flow through the ball valve 210. More specifically, the handle 218 may be used to rotate the ball valve 210 within the spherical chamber 206 to control the amount of flow through the ball valve 210. The angled side walls 211A and 211B of the indentation 211 may be used to stop the over rotation of the handle 218. Thus, the angled side walls 211A and 211B may be used to define the rotation limits of the handle 218.

In accordance with one embodiment, the handle 218 may have a “U” shaped body 218A. A pin member 218B may be formed at the end of each leg of the “U” shaped body 218A. Each pin member 218B may extend inwards from the end of each leg of the “U” shaped body 218A. The pin members 218B may be used to secure the handle 218 to the ball valve 210. Each pin member 218B will be configured to be inserted into a respective socket 214 formed in a corresponding spindle arm 212 thereby securing the handle 218 to the ball valve 210. The configuration of each pin member 218B will be the same as the socket 214 formed in each spindle arm 212. Thus, if the socket 214 has a six-sided interior perimeter, then the pin member 218B will have a six-sided outer perimeter thereby securing the handle 218 to the ball valve 210.

A plurality of slots 220 may be formed in an exterior surface of the handle 218. In accordance with an embodiment, the slots 220 may be formed on a top exterior surface of the handle 218. The slots 220 may be used to provide a grip area to the user that may be used as a slip resistant element on the handle 218.

To prevent leakage, between the channel 204 and the ball channel 210B, an O-ring 222 may be placed at the opening at each transverse end 208A and 208B of the transverse spherical wall 208 of the spherical chamber 206.

Ribbing 224 may be formed on one end of the body 200. The ribbing may be used to secure a hose from a supply to the body 200.

The present invention is not intended to be restricted to any particular form or arrangement, or any specific embodiment, or any specific use, disclosed herein, since the same may be modified in various particulars or relations without departing from the spirit or scope of the claimed invention hereinabove shown and described of which the apparatus or method shown is intended only for illustration and disclosure of an operative embodiment and not to show all of the various forms or modifications in which this invention might be embodied or operated.

What is claimed is:

1. A handle for attachment to a source selected from the group consisting of suction, air, and liquid at a rear end and attached to a tool selected from a dental tool and a medical tool at a front end, the handle for attachment comprising:
an elongated handle valve body defining a channel, the channel running through a length of the elongated handle valve body;
a spherical chamber defined by the elongated handle valve body and positioned within a path of the channel; and
a ball valve rotatably positioned within the spherical chamber.

2. The handle for attachment of claim 1, further comprising an indentation formed in a top surface of the elongated handle valve body.

3. The handle for attachment of claim 2, wherein the indentation has a width that extends across a width of the elongated handle valve body.

4. The handle for attachment of claim 2, wherein a bottom area of the indentation may be formed near a center line of the elongated handle valve body.

5. The handle for attachment of claim 2, wherein the indentation is V-shaped and has two angled side walls.

6. The handle for attachment of claim 5, wherein the two angled side walls of the indentation create a gap along a length of a top surface of the elongated handle valve body.

7. The handle for attachment of claim 1, wherein the spherical chamber comprises:
a transverse spherical wall;
an interior chamber surrounded by the transverse spherical wall; and
two openings formed at opposing transverse ends of the transverse spherical wall.

8. The handle for attachment of claim 1, wherein the ball valve comprises:
a ball defining a ball channel, the ball channel running through a center of the ball;
a pair of spindle arms formed on the ball, the spindle arms being in axial alignment with each other and located on opposite sides of the ball; and
a handle coupled to the spindle arms, the handle for rotating the ball about the spindle arms.

9. The handle for attachment of claim 8, further comprising a socket formed in each spindle arm.

10. The handle for attachment of claim 9, wherein each socket is multi-sided.

11. The handle for attachment of claim 8, wherein the handle of the ball valve is U-shaped and has a pin member formed at each end.

12. The handle for attachment of claim 11, wherein each pin member is configured to be inserted into a corresponding socket formed in a corresponding spindle arm.

13. The handle for attachment of claim 1, wherein the elongated handle valve body further defines a pair of side openings, the side openings being in axial alignment with each other and located on opposite sides of the spherical chamber.

14. The handle for attachment of claim 13, wherein the side openings extend outwardly and laterally in opposite directions from the spherical chamber.

15. A handle for attachment to a source selected from the group consisting of suction, air, and liquid at a rear end and attached to a tool selected from a dental tool and a medical tool at a front end, the handle for attachment comprising:
an elongated handle valve body defining a channel, the channel running through a length of the elongated handle valve body;
a V-shaped indentation formed in a top surface of the elongated handle valve body;
a spherical chamber defined by the elongated handle valve body and extending upwardly from the V-shaped indentation;
a pair of side openings formed on opposite sides of the spherical chamber; and
a ball valve rotatably positioned within the spherical chamber, the ball valve comprising:
a ball defining a ball channel, the ball channel running through a center of the ball; and
a pair of spindle arms formed on the ball, the spindle arms located on opposite sides of the ball and extending out of the pair of side openings; and
a handle coupled to the spindle arms, the handle for rotating the ball about the spindle arms.

16. The handle for attachment of claim 15, wherein the spherical chamber comprises:
a transverse spherical wall;
an interior chamber surrounded by the transverse spherical wall; and
two openings formed at opposing transverse ends of the transverse spherical wall.

17. The handle for attachment of claim 16, wherein the side openings on the spherical chamber are positioned at about 90° from the openings at each transverse end of the transverse spherical wall.

18. The handle for attachment of claim 15, wherein the V-shaped indentation has two angled side walls and wherein rotation of the handle of the ball valve is limited by the two angled side walls.

19. The handle for attachment of claim 15, wherein the handle of the ball valve moves the ball between:
an open position where the ball channel of the ball aligns with the channel of the elongated handle valve body; and
a closed position wherein the ball channel of the ball blocks the channel of the elongated handle valve body.

20. A handle for attachment to a source selected from the group consisting of suction, air, and liquid at a rear end and attached to a tool selected from a dental tool and a medical tool at a front end, the handle for attachment comprising:
an elongated handle valve body;
a channel having a first open end and a second open end running along a length of the elongated handle valve body, the first open end at an opposite end of the elongated handle valve body from the second open end;
a spherical chamber defined by the body and positioned within a path of the channel and having openings at each transverse end;
an indentation formed in a top surface of the body and extending across a width of the elongated handle valve body, wherein the spherical chamber extends up from the indentation; and
a ball valve positioned within the spherical chamber, wherein the ball valve comprises:
a ball;
a ball channel running through a center of the ball and having a pair of open ends;
a pair of spindle arms, wherein each spindle arm is in axial alignment and located on opposite sides of the ball;
a socket formed in each spindle arm;
a handle having a U-shaped body coupled to the ball; and
a pin member formed at an end of each leg of the U-shaped body, each pin inserted into a corresponding socket securing the handle to the ball.
